(19) United States
(12) Patent Application Publication
(10) Pub. No.: US 2005/0165398 A1
(43) Pub. Date: Jul. 28, 2005

(54) Percutaneous spine distraction implant systems and methods

(76) Inventor: Mark A. Reiley, Piedmont, CA (US)

Correspondence Address: RYAN KROMHOLZ & MANION, S.C., POST OFFICE BOX 26618, MILWAUKEE, WI, 53226, US

(21) Appl. No.: 11/041,570

(22) Filed: Jan. 24, 2005

Related U.S. Application Data
(60) Provisional application No. 60/539,208, filed on Jan. 26, 2004.
(60) Provisional application No. 60/600,039, filed on Aug. 9, 2004.

(51) Int. Cl. A61B 17/70; A61B 17/88; A61F 2/44

(52) U.S. Cl. 606/61; 623/17.11; 606/90; 606/73

(57) ABSTRACT

Systems and methods for treating spinal stenosis insert a guide element percutaneously into proximity with the adjacent spinous processes. The systems and methods direct an implant device over the guide element to a position resting between the adjacent spinous processes. The device is sized and configured to distend the adjacent spinous processes. The implant device itself can be variously constructed. It can, e.g., possess threaded lands and/or a notched region in which a spinous process can rest. The implant device has a lumen to accommodate passage of the guide element, so that the device can be passed percutaneously over the guide element for implantation between adjacent spinous processes.

### RELATED APPLICATIONS

This application claims the benefit of provisional U.S. Application Ser. No. 60/539,208, filed Jan. 26, 2004, and provisional U.S. Application Ser. No. 60/600,039, filed Aug. 9, 2004.

### FIELD OF THE INVENTION

The invention generally relates to systems and methods for treating conditions of the spine, and, in particular, systems and methods for distending the spine and/or blocking and/or limiting spinal extension for treating, e.g., spinal stenosis.

### BACKGROUND OF THE INVENTION

Spinal stenosis is a narrowing of the spinal canal. The narrowing of the spinal canal itself does not usually cause any symptoms. However, symptoms such as pain, weakness, and/or numbness appear when the narrowing leads to compression of the spinal cord and nerve roots. The nerves react by swelling and undergoing inflammation.

While spinal stenosis can be found in any part of the spine, the lumbar and cervical areas are the most commonly affected. Patients with lumbar spinal stenosis may feel pain, weakness, or numbness in the legs, calves or buttocks. In the lumbar spine, symptoms often increase when walking short distances and decrease when the patient sits, bends forward or lies down. Cervical spinal stenosis may cause similar symptoms in the shoulders, arms, and legs; hand clumsiness and gait and balance disturbances can also occur. In some patients the pain starts in the legs and moves upward to the buttocks; in other patients the pain begins higher in the body and moves downward. The pain may radiate or may be a cramping pain. In severe cases, the pain can be constant, excruciating, and debilitating.

Some people are born with spinal stenosis. Typically, however, spinal stenosis occurs as the gradual result of aging and “wear and tear” on the spine during everyday activities. The incidence of spinal stenosis increases as people exceed 50 years of age.

Stenosis can sometimes be treated without surgery, e.g., through the use of medications, steroid injections, rest or restricted activity, or physical therapy. In cases when non-surgical treatments are not effective, surgical treatments can be performed, e.g., decompressive laminectomy, laminotomy, foraminotomy, cervical discectomy and fusion, cervical corpectomy, and laminoplasty. The use of surgically implanted devices that distract the spine, called the X-Bar, has also been advocated, e.g., as disclosed in U.S. Pat. No. 6,451,020.

These surgical techniques, though effective for many, are invasive. They require exposure of a section of the spine through an open incision, approximately two inches in length, made along the midline of the back, for excision of vertebral lamina or the placement of an implant between adjacent spinous processes. Due to the obvious risks involved, many surgeons will not consider open surgical treatment of spinal stenosis unless several months of non-surgical treatment methods have been tried.

### SUMMARY OF THE INVENTION

The present invention overcomes the problems and disadvantages associated with current strategies and systems in the treatment of spinal stenosis by invasive, open surgical procedures.

One aspect of the invention provides systems and methods for treating spinal stenosis. The systems and methods direct an implant device to a position resting between the adjacent spinous processes. The device is sized and configured to distend the adjacent spinous processes. The device can also block or limit extension of the back. The device includes a region that, in use, receives a spinous process. The region tapers from a high surface to a low surface in an anterior-to-posterior direction.

Other objects, advantages, and embodiments of the invention are set forth in part in the description which follows, and in part, will be obvious from this description, or may be learned from the practice of the invention.

### DESCRIPTION OF THE DRAWINGS

FIG. 1 shows a vertebra with a normal neuroforamen.

FIG. 2 shows the passage of the spinal cord and nerve roots in a normal neuroforamen.

FIG. 3 shows a vertebra with a stenotic neuoforamen, i.e., a neuroforamen that has a reduced sized, compared to the neuroforaman shown in FIG. 1.

FIG. 4 shows the narrowing of the spaces in the spine that results in pressure on the spinal cord and/or nerve roots, causing nerves to swell and become inflamed.

FIG. 5 shows a device that has been implanted by percutaneous access between adjacent first and second spinous processes of the stenotic vertebrae shown in FIG. 4 to relieve the pressure on the spinal cord and/or nerve roots.

FIG. 6 shows the device shown in FIG. 5 as it exists outside the body, prior to implantation.

FIGS. 7 to 12 show the implantation of the device shown in FIG. 6 by percutaneous access.

FIG. 13 shows an alternative embodiment of a device that can be implanted by percutaneous access between adjacent first and second spinous processes of the stenotic vertebrea to relieve the pressure on the spinal cord and/or nerve roots.

FIGS. 14 to 16 show the implantation of the device shown in FIG. 13 by percutaneous access.

FIG. 17 shows the device shown in FIG. 13 after implantation.

FIG. 18A shows an alternative embodiment of a device that can be implanted by percutaneous access between adjacent first and second spinous processes of the stenotic vertebrea to relieve the pressure on the spinal cord and/or nerve roots.

FIG. 18B is a section view of the device taken generally along line 18B-18B in FIG. 18A.

FIG. 19 shows the implantation of the device shown in FIG. 18A by percutaneous access.

FIG. 20 shows the device shown in FIG. 18A after implantation.

FIG. 21 is a section view of the device taken generally along line 21-21 in FIG. 20.

FIGS. 22A and 22B are perspective views of an alternative embodiment of a device that can be implanted by percutaneous access between adjacent first and second spinous processes of the stenotic vertebrea to relieve the pressure on the spinal cord and/or nerve roots and having a hinge mechanism and in which the angle of the inclined planes may be controlled by a series of screws and bores.

FIG. 23 is a side view of the device of FIGS. 22A and 22B implanted between adjacent first and second spinous processes of the stenotic vertebrae and in a contracted condition.

FIG. 24 is a side view similar to FIG. 23 and illustrating the device in an enlarged condition which relieves pressure on the spinal cord and/or nerve roots.

FIG. 25 is a top plan view of the bottom arm of the device of FIGS. 22A and 22B illustrating a configuration and placement of screws and bores which serves to raise the incline planes of the device upon insertion of the screws into the bores.

FIG. 26 is a view similar to FIG. 25 and illustrating an alternative configuration and placement of screws and bores.

FIG. 27 is a perspective view illustrating the device of FIGS. 22A and 22B implanted between adjacent first and second spinous processes after insertion of screws into the bores and the incline planes raised.

FIG. 28 is a view similar to FIG. 25 and illustrating another alternative configuration and placement of screws and bores.

FIG. 29 is a view similar to FIG. 25 and illustrating another alternative configuration and placement of screws and bores.

FIG. 30 is a side view of an alternative embodiment of a device that can be implanted by percutaneous access between adjacent first and second spinous processes of the stenotic vertebrea to relieve the pressure on the spinal cord and/or nerve roots and having a hinged mechanism and in which the angle of the inclined planes may be controlled by an enlargeable bladder.

FIG. 31 is an alternative embodiment of the device of FIG. 30.

FIG. 32 is a bottom plan view of the upper arm of an alternative embodiment of the device of FIG. 30 having left and right bladders.

FIG. 33 is a bottom plan view of the upper arm of an alternative embodiment of the device of FIG. 30 having anterior and posterior bladders.

### DESCRIPTION OF THE PREFERRED EMBODIMENT

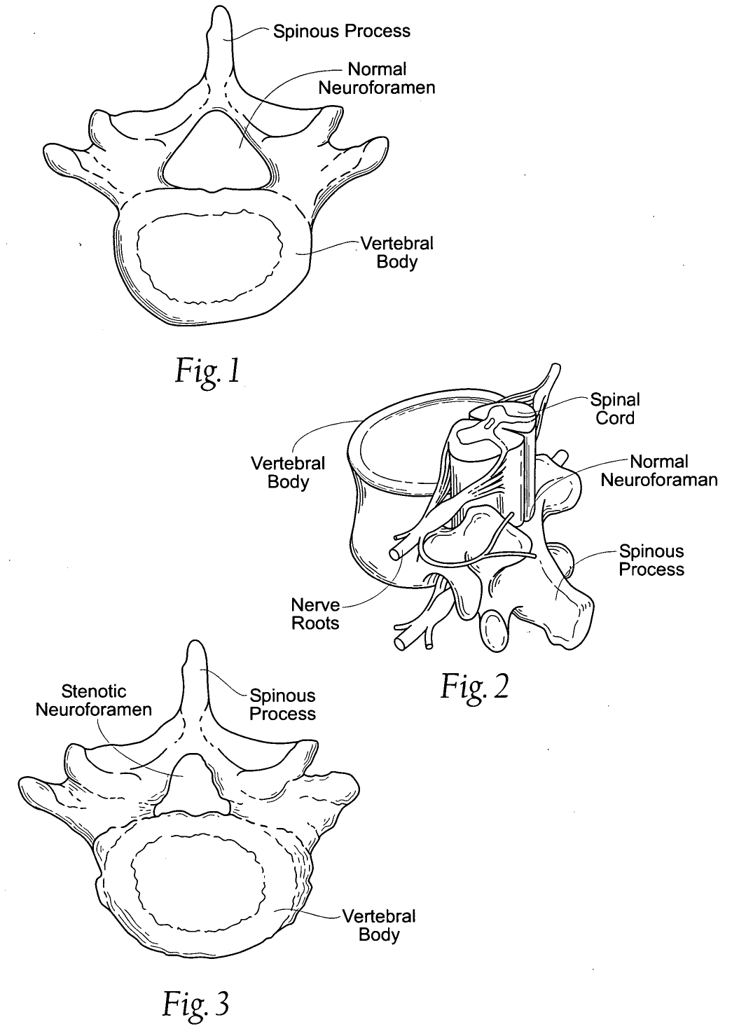

FIG. 1 shows a vertebra with a normal neuroforamen. FIG. 2 shows the passage of the spinal cord and nerve roots in a normal neuroforamen.

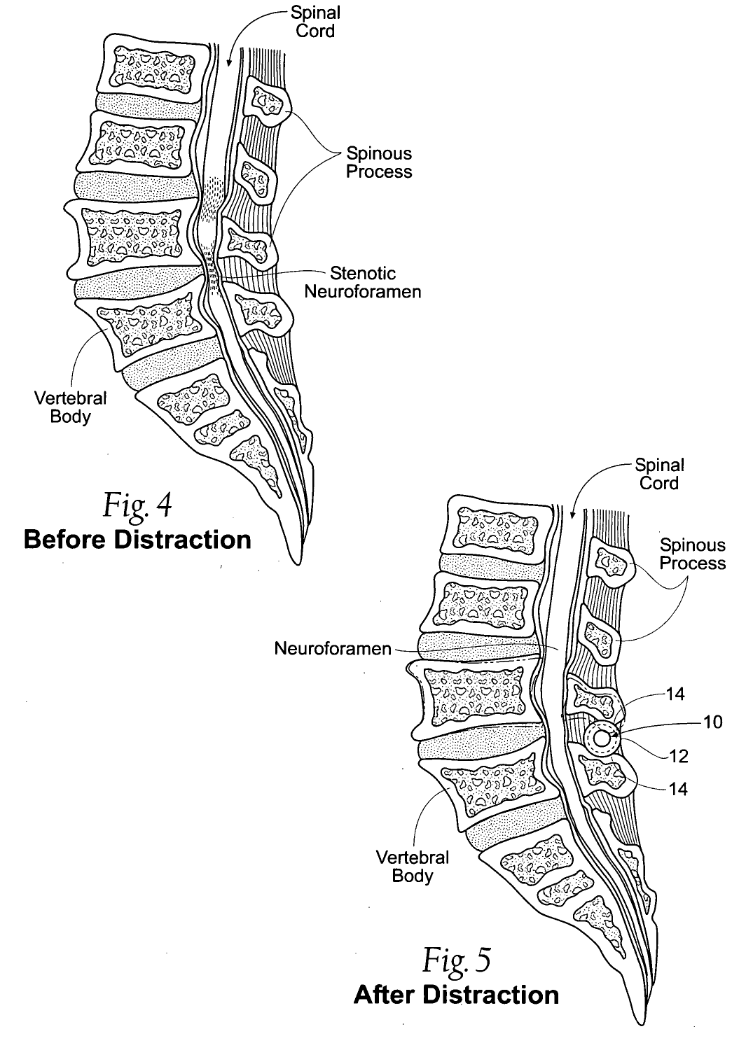

FIG. 3 shows a vertebra with a stenotic neuoforamen, i.e., a neuroforamen that has a reduced sized, compared to the neuroforaman shown in FIG. 1. As FIG. 4 shows, the narrowing of the spaces in the spine that results in pressure on the spinal cord and/or nerve roots. When the neuroforamina are reduced in size, the nerves may swell and become inflamed, causing pain and discomfort.

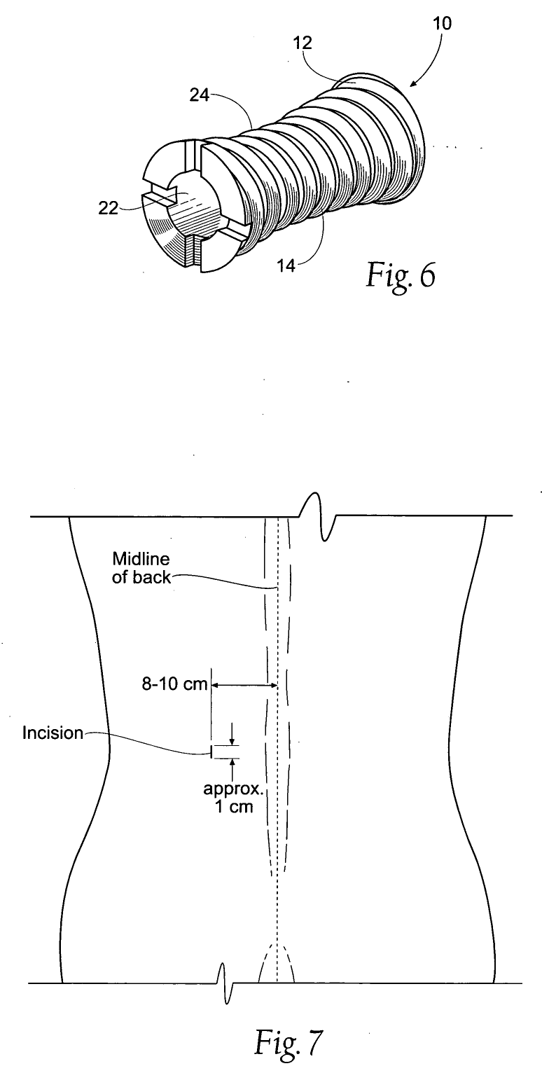

FIG. 5 shows a device 10 that has been implanted by percutaneous access between adjacent first and second spinous processes of the stenotic vertebrea shown in FIG. 4. The device 10 relieves the pressure on the spinal cord and/or nerve roots. FIG. 6 shows the device 10 as it exists outside the body, prior to implantation. The device 10 can be made of a durable prosthetic material, such as, e.g., polyethylene, rubber, a sponge material (e.g., polyethylene sponge), tantalum, titanium, chrome cobalt, surgical steel, bony in-growth material, ceramic, artificial bone, or a combination thereof.

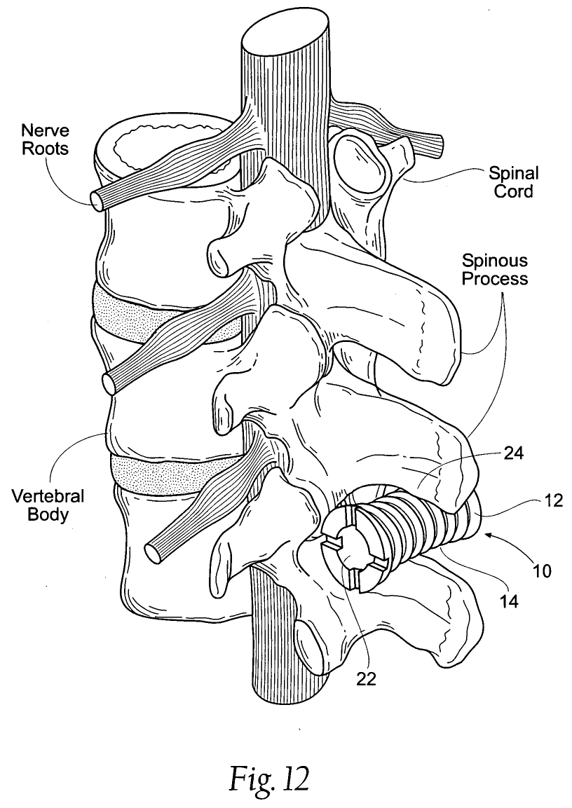

The implanted device 10 includes a body 12 having a contact region 14 that, in use, rests between the first and second spinous processes (see FIG. 12). As FIG. 12 best shows, the region 14, in use, engages both spinous processes to apply a separating force. The force spreads apart or distracts the spinous processes.

The degree of distraction can be seen by comparing FIG. 5 (with distraction) with FIG. 4 (before distraction). The distraction enlarges the volume of the spinal canal to alleviate pressure on blood vessels and/or nerves, thereby treating the pain and other symptoms that can accompany spinal stenosis.

In use, the implanted device 10 also serves as an extension stop for the back. As the back is bent backwardly and placed in extension, the presence of the implanted device 10 resists extension beyond a given point. Due to the presence of the implanted device 10, the spacing between adjacent spinous processes cannot be reduced to less than the outside diameter. of the body region 14. Typically, given an outside diameter of between 5 mm to 14 mm, the presence of the implanted device 10 can serve to block the last 4° to 5° of extension. Pressure on nerves and the resulting pain are therefore alleviated or reduced.

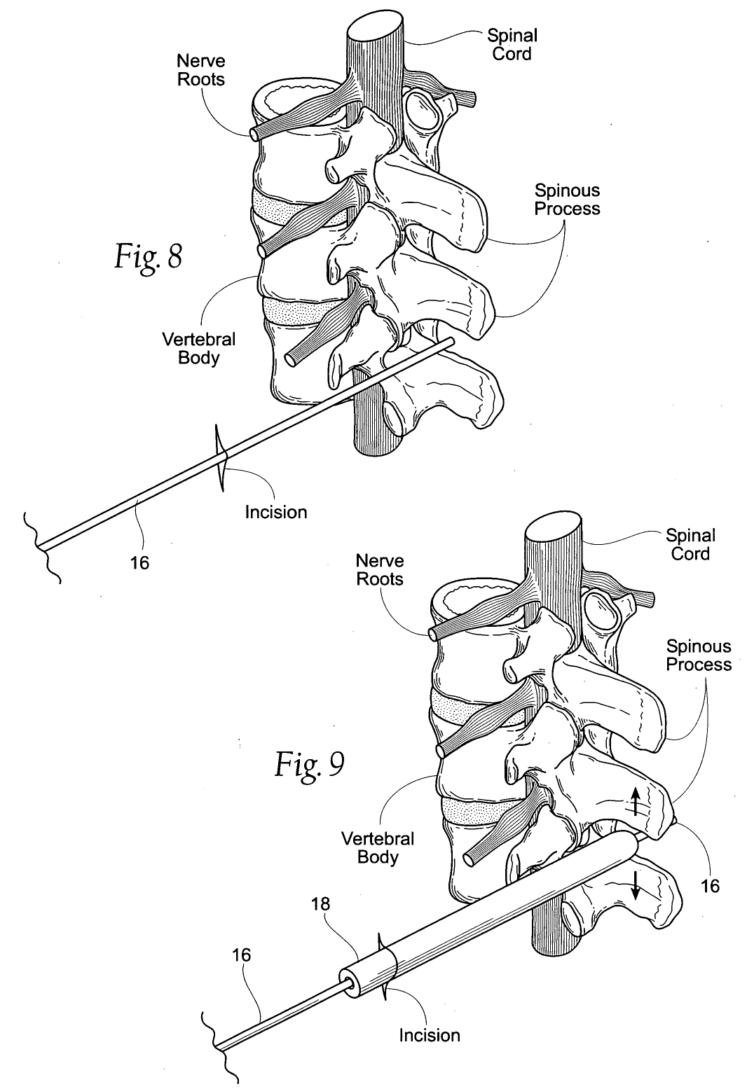

Significantly, the device 10 can be implanted by non-invasive percutaneous access, instead of requiring an open surgical procedure. As FIG. 7 shows, a small incision, e.g., 1 cm, is desirably made about 8 cm to 10 cm from the midline of the back. With reference to FIG. 8, a guide pin 16 is inserted through the incision. Under imaging guidance (e.g., x-ray (fluoroscopy), ultrasound, magnetic resonance, computed tomography, or combinations thereof) the guide pin 16 is inserted in between the adjacent spinous processes.

A first tubular obturator 18 is inserted over the guide pin 16 under imaging guidance into the space between the two spinous process (see FIG. 9). The outside diameter of the obturator 18 is selected to initiate distension of the spinous processes.

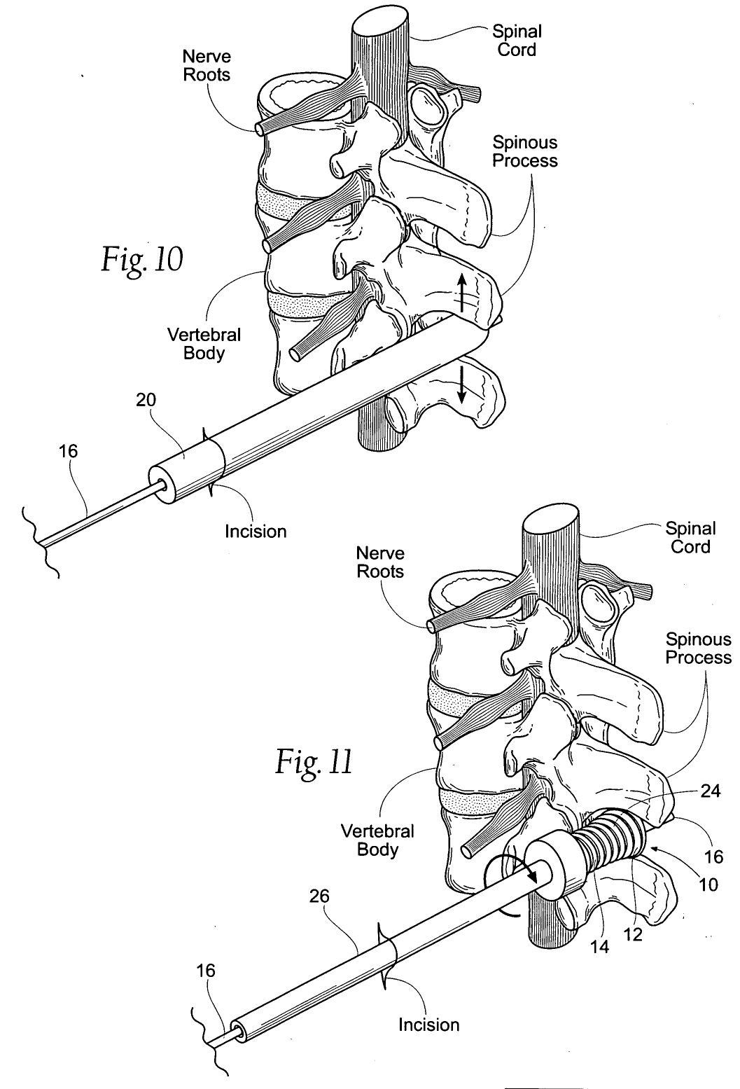

The first tubular obturator 18 is withdrawn over the guide pin 16, and a second tubular obturator 20 is inserted over the guide pin 16 under imaging guidance into the previously distended space between the spinous processes (see FIG. 10). The second tubular obturator 20 has a second outside diameter greater than the outside diameter of the first obturator 18, to open a greater distention of the spinous processes. This distension is slightly smaller than the outside diameter of the body region 14 of the device 10 to be implanted. The second obturator 20 is then withdrawn over the guide pin 16. Additional (or fewer) obturators may be deployed in this manner until a. desired degree of distension is achieved.

The device 10 is now inserted over the guide pin 16 under imaging guidance into the distended space between the spinous processes (FIG. 11). As FIG. 6 shows, the body 12 of the device 10 includes an interior lumen 22 to accommodate its passage over the guide pin 16.

The body 12 of the device 10 can be sized and configured in various ways. The body 12 can, e.g., be cylindrical, square, rectangular, or curvilinear (banana-shaped). The body 12 also desirably includes threaded lands 24, so that the device 10 functions as a screw. A screw driving tool 26 passes over the guide pin 16 and engages the device 10 (FIG. 11), to rotate the device 10 about the guide pin 16 and advance the device 10 between the spinous processes. The threaded lands 24 take purchase in the bone of the spinous processes, to secure the device 10 in place between the distended spinous processes.

The tool 26 and guide pin 16 can now be withdrawn, leaving the implanted device 10 behind (FIG. 12). The incision is closed. The implantation of the device 10 has been completed percutaneously and without need of an open surgical procedure.

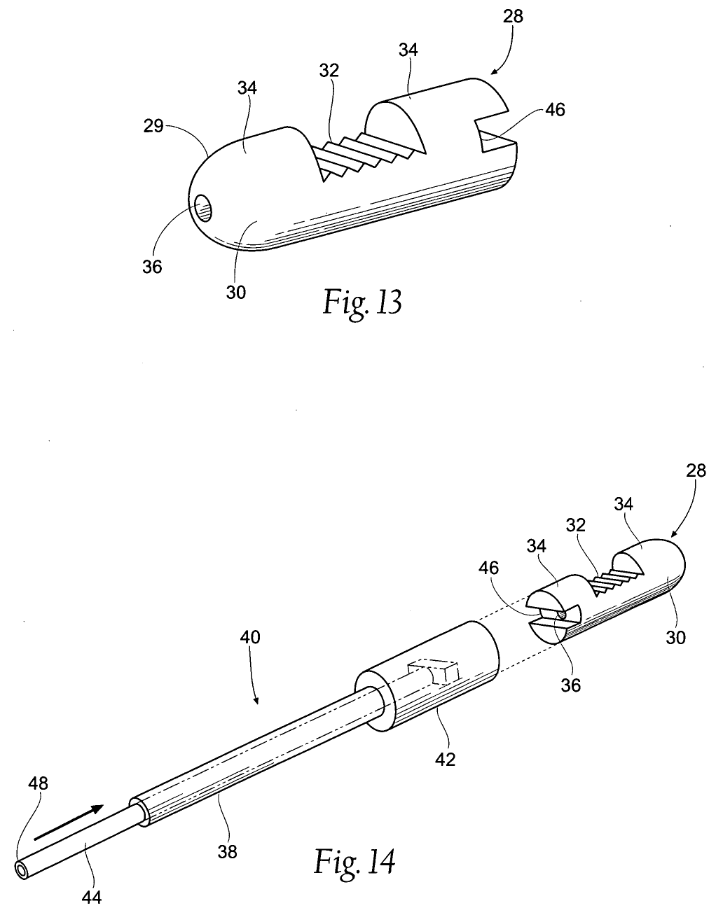

FIG. 13 shows an alternative embodiment of a device 28 that can be implanted by percutaneous access to cause distention between adjacent first and second spinous processes of stenotic vertebrea. The device 28 includes a blunt nose 29 and a bullet-shaped body 30 having a stepped-down or notched region 32 between adjoining stepped-up or ridge regions 34. Desirably, the interior of the notched region 32 includes grooves, lands, or an otherwise roughened exterior surface to gain purchase in bone.

Like the body 12, the body 30 can be made of a durable prosthetic material, such as, e.g., polyethylene, rubber, a sponge material (e.g., polyethylene sponge), tantalum, titanium, chrome cobalt, surgical steel, bony in-growth material, ceramic, artificial bone, or a combination thereof. Also like the body 12, the body 30 includes a lumen 36 to accommodate passage of a guide pin 16, as will be described in greater detail later.

In a typical embodiment, the body 30 measures about 9 mm in overall length, and the regions 32 and 34 are approximately equal in length (i.e., each being approximately 3 mm in length). The outside diameter of the body 30 at the ridge regions 34 can be about 5 mm to 6 mm. The depth of the notched region 32 can be about 2 mm. If desired, there can be two, oppositely facing notched regions 32 (not shown).

As FIG. 14 shows, the device 28 is desirably implanted using a tool 40 that comprises a sleeve 42 carried at the end of a handle 38 and a pusher 44 that entends through the handle 38 into the sleeve 42. The sleeve 42 accommodates insertion of the device 28, with its blunt distal end partially exposed. The pusher 44 serves, in use, to push against the proximal end of the device 28 within the sleeve 42, to expel the device 28 from the sleeve 42. The proximal end of the body 30 desirably includes a receptacle 46 in which the pusher 44, when in use, rests. The pusher 44 includes a lumen 48 that accommodates passage of a guide pin 16, so the tool 40, like the device 28 can be percutaneously deployed.

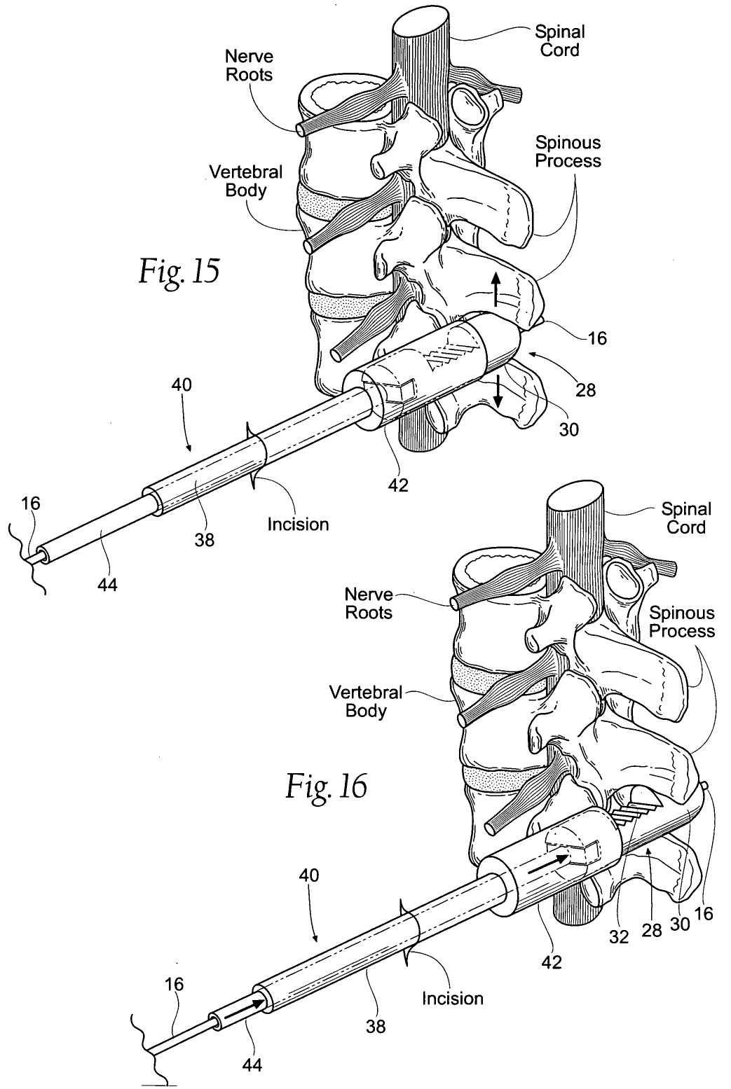

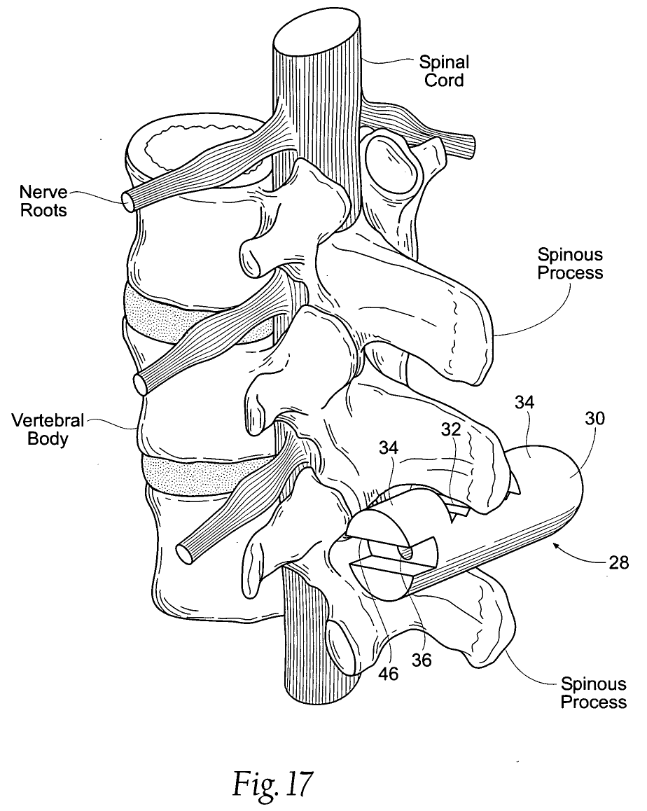

In use, the guide pin 16 and obturators 18 and 20 are manipulated under imaging guidance as previously described and shown in FIGS. 7 to 10. At this point in the procedure (see FIG. 15), the tool 40, carrying the device 28 (the device 28 being preferably retracted, at least in part, within the sleeve 42), is deployed over the guide pin 16 to a location adjacent the distended spinous processes. The pusher 44 is advanced forward (see FIG. 16), to expel the device 28 from the sleeve 42. The blunt distal end of the body 30 enters the distended space between the processes, distending them slightly more, until one of the spinous processes settles within the notched region 32(see FIG. 17) (if two notched regions are present, both spinous processes will settle into its own notched region). The tool 40 is withdrawn back over the guide pin 16. The guide pin 16 is removed, leaving the device 28 resting between the two spinous processes. The incision is closed. The percutaneous implantation of the device 28 has been completed.

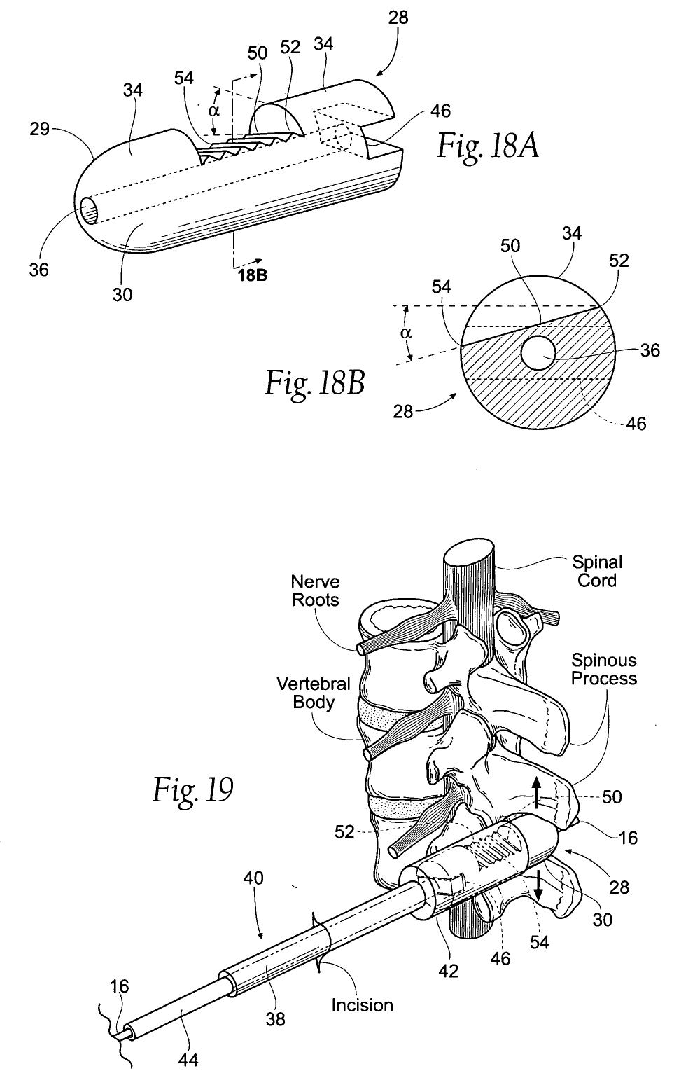

FIGS. 18A and 18B show an alternative embodiment of the device 28. Structural elements that are shared with the device 28 shown in FIGS. 13 are designated by the same reference numbers. In FIGS. 18A and 18B, the device 28 includes a notched region 50, where the spinous process rests when the device 28 is installed. Unlike the notched region 32 in FIG. 13, the notched region 50 is tapered between a high surface 52 and a low surface 54. In a representative embodiment, the taper forms an angle α (shown in FIGS. 18A and 18B) that is in the range of 4-degrees to 25-degrees from horizonal, which is gauged relative to the anterior-to-posterior orientation of the receptacle 46. If desired, there can be two, oppositely facing notched tapered notched regions 32 (not shown). As with the notched region 32, the interior of the notched region 50 can include grooves, lands, or otherwise roughened exterior surface to gain purchase in bone.

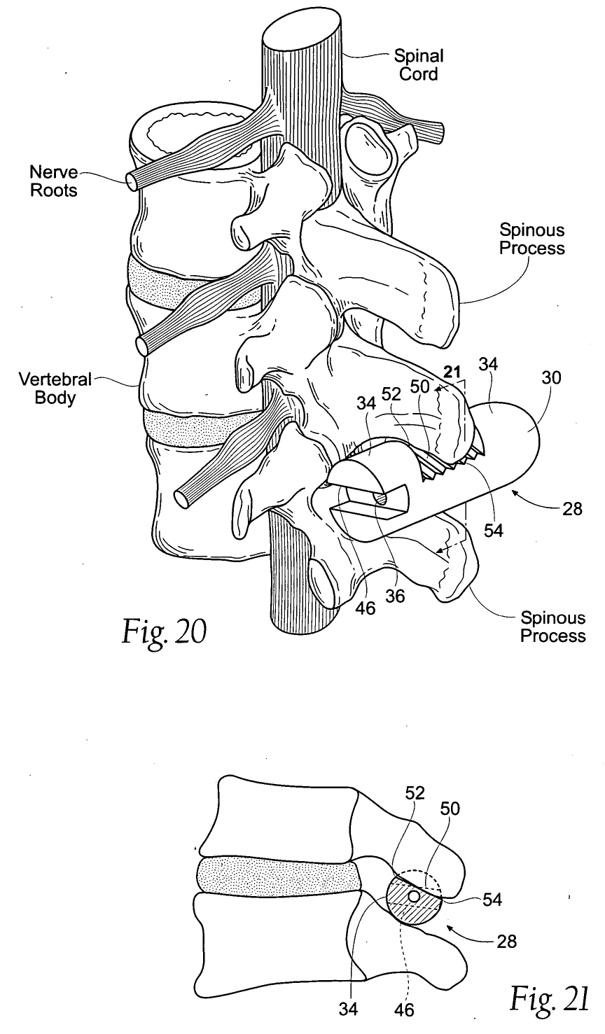

In use, the device 28 is installed between adjacent first and second spinous processes of stenotic vertebrae (see FIG. 20), such that the high surface 52 is oriented in an anterior direction—i.e., adjacent the disc—and the low surface 54 is oriented in a posterior direction—i.e., facing away from the vertebral body (see FIG. 21, also).

The taper angle α of the notched region 50 is preferably selected to approximate the degree of the posterior curvature of the spinous process that settles within the notched region 50, to maximize contact between the notched region 50 and the spinous process throughout the notched region 50. The degree of taper may be chosen to accommodate a specific location and/or individual anatomy. The inferior side of the device 28 can also be notched in the same manner with a posterior-directed taper 52, so that spinous processes will settle into the superior and interior notched regions 50.

To install, the guide pin 16 and obturators 18 and 20 are manipulated under imaging guidance as previously described and shown in FIGS. 7 to 10. The tool 40, carrying the device 28 (the device 28 being preferably retracted, at least in part, within the sleeve 42), is deployed over the guide pin 16 to a location adjacent the distended spinous processes such that the tapered region 50 is oriented with the high surface 52 directed anteriorly and the low surface 54 directed posteriorly, as shown in FIG. 19. The pusher 44 is then advanced forward to expel the device 28 from the sleeve 42, as previously described.

The blunt distal end 29 of the body 30 enters the distended space between the processes, distending them slightly more, until one (or both, depending upon the configuration) of the spinous processes settles within the notched region 50, as shown in FIGS. 20 and 21.

Distraction of stenotic vertebrae may also be accomplished by placement of an enlargeable or expandable structure between adjacent first and second spinous processes. The enlargeable structure may be selectively manipulated between a contracted condition suitable for percutaneous introduction between the spinous processes and an expanded or enlarged condition in which the expandable structure engages both spinous processes to apply a separating force to spread apart or distract the spinous processes. The enlargeable structure may take various configurations suitable for percutaneous access and providing suitable distraction. By way of example and not limitation, a representative embodiment will now be described.

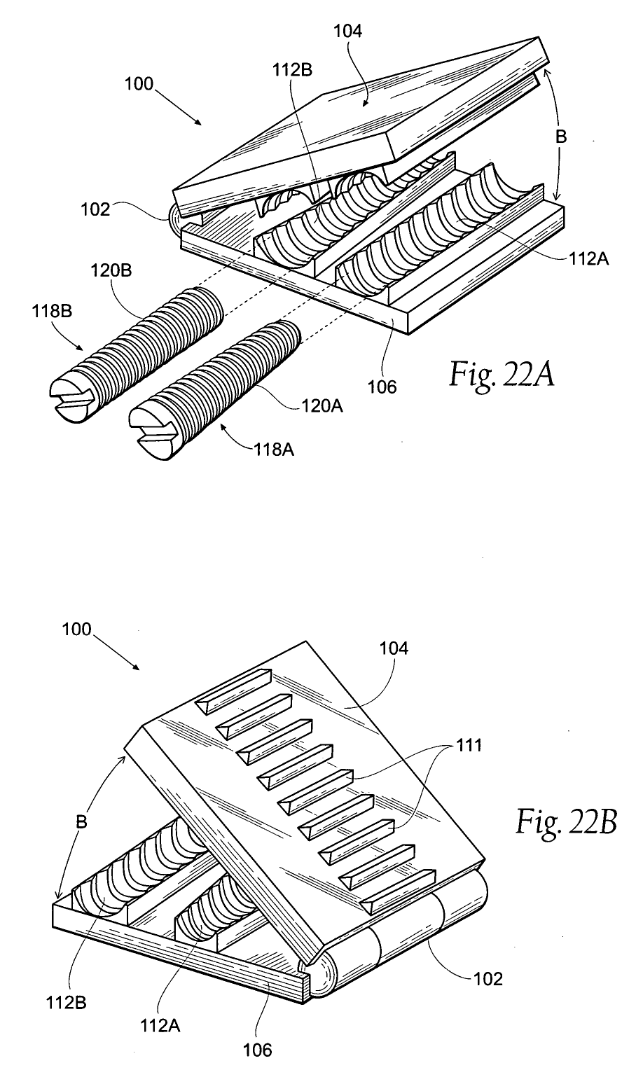

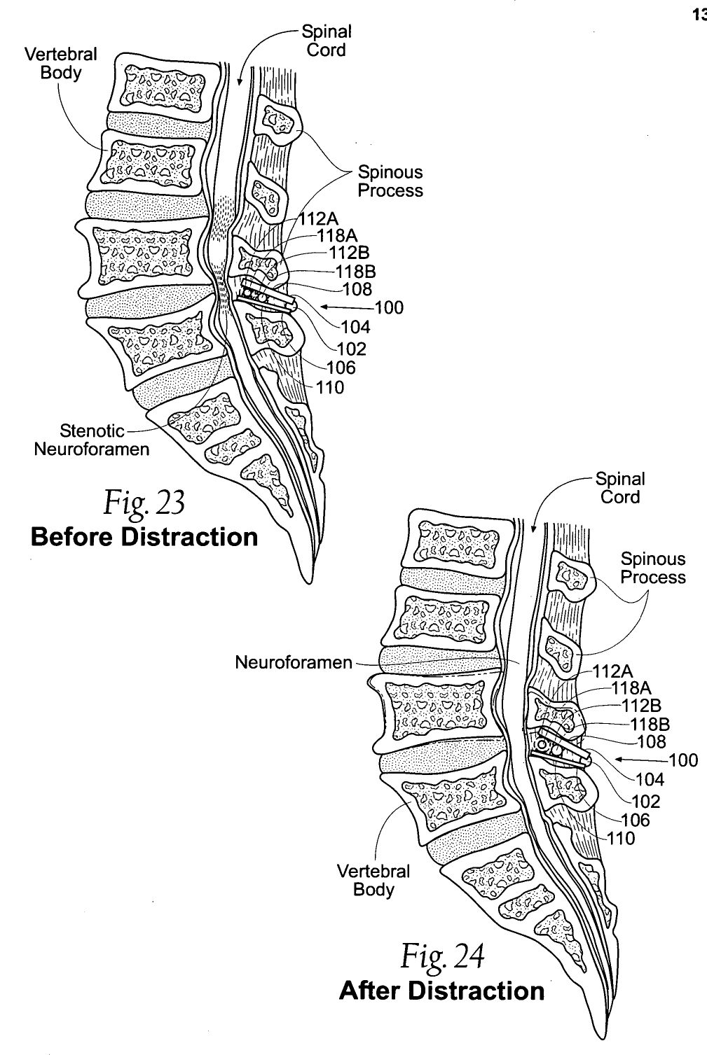

FIG. 22A shows a device 100 suitable for non-invasive insertion by percutaneous access and without requiring an open surgical procedure. The device 100 provides a hinged arrangement that permits selective expansion of the device 100 to allow adjustment of incline planes to the desired angle for each interspinous process. The device 100 has a contracted condition, shown in FIG. 23, suitable for percutaneous insertion between adjacent spinous processes and an expanded condition, shown in FIG. 24, in which the device 100 engages both spinous processes to apply a separating force to spread apart or distract the spinous processes.

As FIGS. 22A shows, the device 100 comprises a hinge 102, a top or first arm 104 and a bottom or second arm 106. The arms 104 and 106 define an angle of taper (β). The arms may be selectively expanded to increase the angle β to a desired angle to accommodate the angle of adjacent spinous processes at a given location on the spinal column and to accommodate individual anatomy.

With reference to FIG. 23, the device 100 is introduced in the contracted condition between adjacent first and second spinous processes of stenotic vertebrae such that the arms 104 and 106 are oriented in an anterior direction, i.e., adjacent the disc, and the hinge 102 is oriented in a posterior direction, i.e., facing away from the vertebral body.

As best seen in FIG. 24, the first arm 104 provides a first contact surface 108 that, upon expansion, engages the first spinous process. The second arm 106 provides a second contact surface 110 that, upon expansion, engages the second spinous process. The contact surfaces 108 and 110 may be essentially smooth, as seen in FIG. 22A. Alternatively, either or both of the contact surfaces 108 and 110 may be roughened or saw-toothed to provide a series of projections 111 in a manner that prevents slippage of the device, as seen in FIG. 22B. The projections 111 may take any of a variety of configurations (e.g., ridges, teeth). It is contemplated that the number, size, and configuration of the projections 111 may be varied as desired or as necessary to prevent slippage.

The device 100 can be made of a durable prosthetic material, such as, e.g., polyethylene, rubber, a sponge material (e.g., polyethylene sponge), tantalum, titanium, chrome cobalt, surgical steel, bony in-growth material, ceramic, artificial bone, or a combination thereof.

The device 100 may be inserted by percutaneous access as previously described and using suitable surgical tools.

In use, the implanted device 100 also serves as an extension stop for the back and can serve to block the last 4° to 5°. Due to the presence of the implanted device 100, the spacing between adjacent spinous processes cannot be reduced to less than angle β. Pressure on nerves and the resulting pain are therefore alleviated or reduced.

A series of complementary and mating fixation members, e.g., screws, and fixation member receivers, e.g., holes or bores, allow for controlled expansion and independent right and left side adjustment to achieve desired inclined planes and thereby create the desired angle β for each interspinous process.

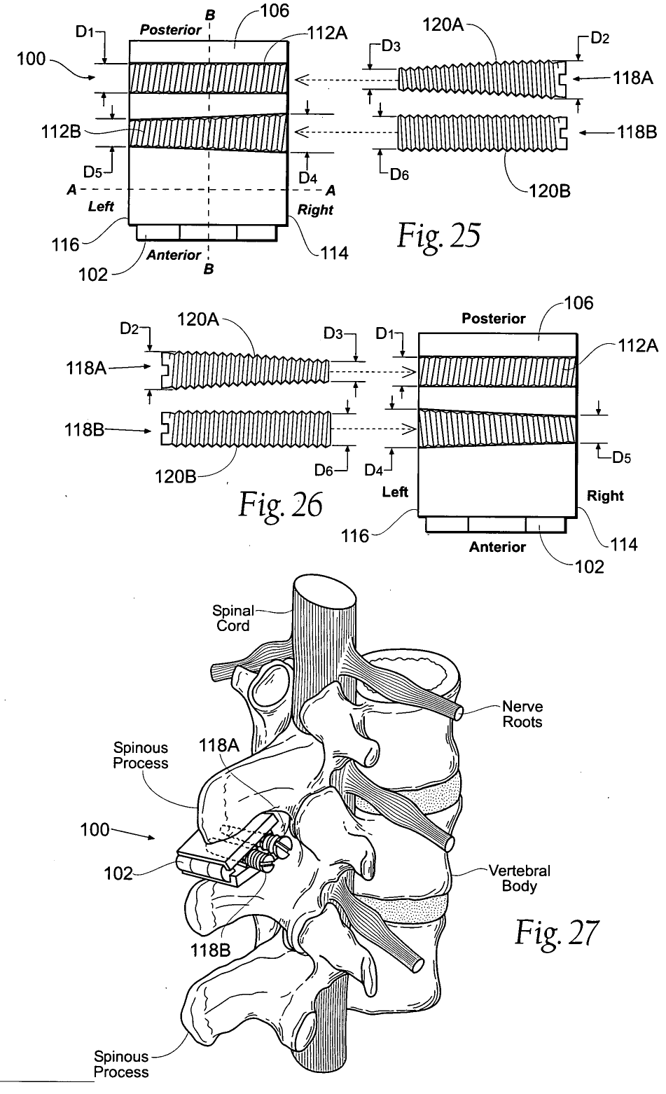

In a representative embodiment illustrated in FIG. 25, a first bore 112A extends in a lateral direction across the spinous processes (i.e., along an axis A and at approximately a 90-degree angle from the axis B of the device 100) from a first side 114 (i.e., the right side in FIG. 25) to a second side 116 (i.e, the left side in FIG. 25) of the device 100 and is of an essentially constant diameter (D1). The first bore 112A receives a first screw 118A, e.g., by threaded engagement. The first screw has a body 120A that tapers medially from the first side 114 to the second side 116 from a larger diameter D2 to a smaller diameter D3. D2 is greater than D1 (D2>D1) such that, upon insertion into the first bore 112A, the first screw 112A raises the first side 114 (i.e., the side of insertion) of the inclined plane formed by the first and second arms 104 and 106 (see also FIG. 27).

A second bore 112B extends in a lateral direction and tapers in diameter medially from a larger diameter D4 to a smaller diameter D5. The second bore 112B receives a second screw 118B e.g., by threaded engagement. The second screw 118B has a body 120B of an essentially constant diameter (D6). D6 is greater than D5 (D6>D5), such that upon insertion into the second bore 112B, the second screw 118B raises the second (i.e., opposing) side 116 of the inclined plane formed by the first and second arms 104 and 106.

The screws may be formed of any suitable durable and biocompatible material, e.g., titanium, titanium alloys, tantalum, chrome cobalt, surgical steel, ceramic, sintered glass, artificial bone, or combinations thereof.

The size as well as the depth of insertion of the screws 118A and 118B can be selectively controlled to achieve the desired incline plane for a given location on the spinal column and to accommodate individual anatomy.

In a representative embodiment, the range of incline plane is adjustable from approximately 4-degrees to approximately 25-degrees from horizontal, which is gauged relative to the anterior-to-posterior orientation of the device 100.

In this arrangement, the first and second screws 118A and 118B are inserted from the same side 114. In the embodiment illustrated in FIG. 25, both screws are inserted from the right or first side 114 such that the first screw 118A raises right side and the second screw 118B raises left or second side 116.

Alternatively, both the first and second screws 118A and 118B may be inserted from the opposing or left side 116, as shown in FIG. 26. In this embodiment, the first screw 118A raises the second or left side 116, while the second screw 118B raises the first or right side 114.

In alternative embodiments, the first and second screws are inserted from opposite sides 114 and 116 respectively. In one embodiment, illustrated in FIG. 28, both the first and second bores 112A and 112B extend in a lateral direction and are of an essentially constant diameter D1 such that the bores 112A and 112B are generally parallel. The bodies 120A and 120B of the first and second screws 118A and 118B, respectively, taper medially from a larger diameter D2 to a smaller diameter D3. The first screw 118A is inserted from the first side 114 to raise the first side 114. The second screw 118B is inserted from the second side 116 to raise the second side 116.

In another embodiment, illustrated in FIG. 29, both of the first and second bores 112A and 112B extend in a lateral direction and taper in diameter medially from a larger diameter D4 to a smaller diameter D5. The first bore 112A tapers medially from the first side 114 toward the second side 116. The second bore 112B tapers medially from the second side 116 toward the first side 114. Both of the first and second screws 118A and 118B have a body 120A and 120B, respectively, of an essentially constant diameter D6. The first screw 118A is inserted from the first side 114 to raise the second (i.e., opposite) side 116. The second screw 118B is inserted the second side 116 to raise the first (i.e., opposite) side 114.

It will be readily apparent to one of skill in the art in view of this disclosure that the number, configuration, and placement of screws 118 and bores 112 may be varied to accommodate specific needs as well as to accommodate individual anatomy.

In other alternative embodiments, an enlargeable container is used to displace or raise the arms 104 and 106 and thereby increase the inclined planes to the desired angle β. For example, FIG. 30 illustrates an alternative embodiment of a device 200 suitable for non-invasive insertion by percutaneous access and without requiring an open surgical procedure. The device 200 has a hinged arrangement and shares features of the device 100 previously described. Therefore, like reference numbers will be assigned to denote like parts.

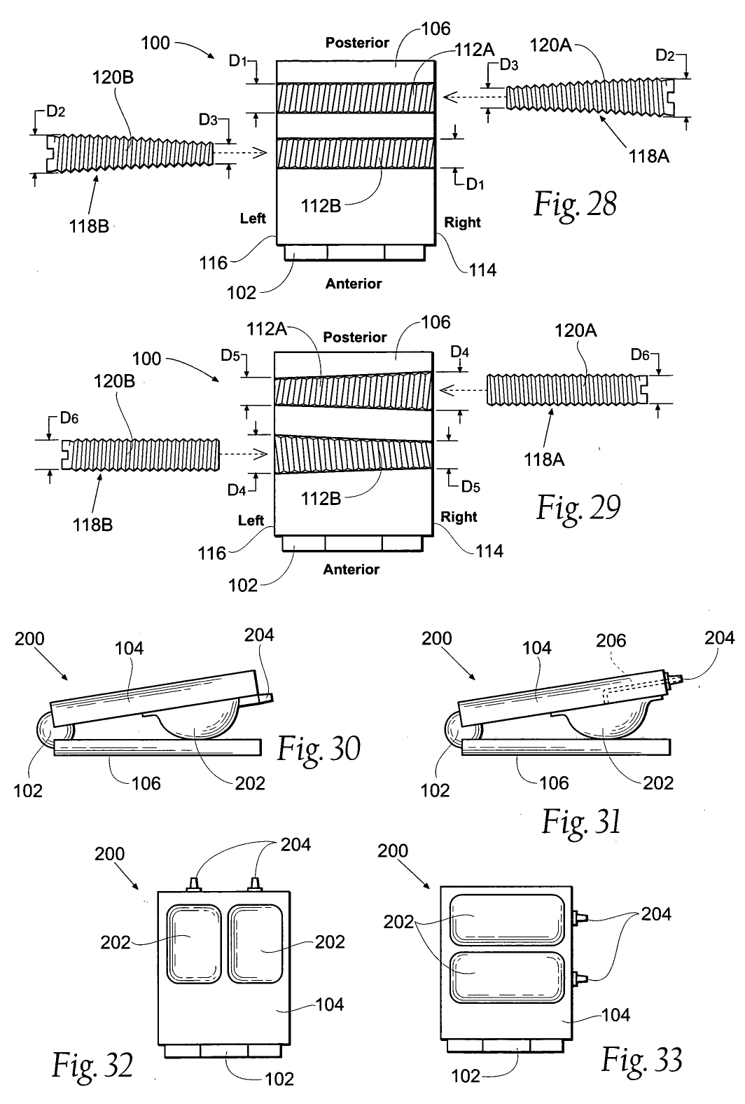

In the illustrated embodiment, a bladder 202 may be inserted between the arms 104 and 106 and expanded or inflated, e.g., by bone cement, to raise the arms 104 and 106 to the desired inclined planes. The bladder 202 may be formed integral with the device 202. The device 200 is inserted between adjacent spinous processes as previously described with the bladder 202 in the contracted condition. As shown in FIG. 30, the bladder 202 may include an injection port 204 for introducing bone cement or other medium into the bladder 202 to enlarge the bladder 202. The degree of expansion of the bladder 202 may be selectively controlled and is desirably uniform in the medial-lateral direction to provide equivalent right and left side distraction. In another embodiment, illustrated in FIG. 31, the arm 104 includes an inflation port 204 that communicates with the bladder 202 through a lumen 206 to permit introduction of a medium into the bladder 202.

Alternatively, the bladder 202 may be a separate component from the device 200. In this arrangement, the device 202 is first inserted between adjacent spinous processes as previously described. The bladder 202 is then inserted in the contracted condition and positioned between arms 104 and 106. A medium is then injected or otherwise introduced into the bladder 202 to enlarge the bladder 202, as previously described.

It is contemplated that multiple bladders 202 can be used, e.g., left and right bladders 202 (FIG. 32), or anterior and posterior bladders (FIG. 33). Desirably, the bladders 202 may be enlarged independently, e.g. by distinct inflation ports 204, to selectively control the degree of enlargement of each bladder 202 to produce the desired angle β. It is further contemplated that the bladders 202 may be of varying size and configuration as desired to accommodate specific needs and individual anatomy.

Other embodiments and uses of the inventions described herein will be apparent to those skilled in the art from consideration of the specification and practice of the inventions disclosed. All documents referenced herein are specifically and entirely incorporated by reference. The specification should be considered exemplary only with the true scope and spirit of the invention indicated by the following claims. As will be easily understood by those of ordinary skill in the art, variations and modifications of each of the disclosed embodiments can be easily made within the scope of this invention as defined by the following claims.

1. An implant device for distending adjacent spinous processes comprising
a body sized and configured to rest between and distend the adjacent spinous processes, the body including inclined planes that taper from a superior surface to an inferior surface in an anterior-to-posterior direction.

2. A device according to claim 1, further including
a lumen in the body sized and configured to accommodate passage of a percutaneous guide element.

3. A device according to claim 1
wherein the inclined planes define a region that is sized and configured to receive a spinous process.

4. A device according to claim 3
wherein the region has a taper angle in the range of 4-25°.

5. A device according to claim 3
wherein the region includes a surface sized and configured to frictionally engage bone.

6. A device according to claim 1
wherein the body is made of at least one selected prosthetic material.

7. A device according to claim 6
wherein the selected prosthetic material includes polyethylene, rubber, tantalum, titanium, chrome cobalt; surgical steel, bony in-growth material, ceramic, artificial bone, or a combination thereof.

8. A device according to claim 1, further including
a tool sized and configured to engage the device and urge the device into a position resting between the adjacent spinous processes, the tool including a lumen accommodating passage of a guide element.

9. A device according to claim 1
wherein the body comprises a hinge mechanism and first and second arms coupled to the hinge mechanism which define an angle of taper.

10. A device according to claim 9
wherein the arms have a contracted condition permitting insertion of the device between adjacent spinous processes and an enlarged condition which distends the adjacent spinous processes.

11. A device according to claim 10
wherein the arms define an angle of taper selectively adjustable between 4-25°.

12. A device according to claim 10, further comprising
means for moving the arms from the contracted position to the enlarged condition to increase the angle of taper.

13. A device according to claim 10, further comprising
a fixation member which engages a fixation member receiver to move the arms from the contracted position to the enlarged condition to increase the angle of taper.

14. A device according to claim 10, further comprising
a bladder adapted to receive an enlarging medium which enlarges the bladder to move the arms from the contracted position to the enlarged condition to increase the angle of taper.

15. A device according to claim 14
wherein the enlarging medium is bone cement.

16. A method for treating spinal stenosis comprising
directing a device as defined in claim 1 to a position resting between the adjacent spinous processes, the device being sized and configured to distend the adjacent spinous processes.
